(19) United States
(12) Patent Application Publication
(10) Pub. No.: US 2018/0126021 A1
(43) Pub. Date: May 10, 2018

(54) STERILIZATION SYSTEM AND METHOD

(71) Applicant: Covidien LP, Mansfield, MA (US)

(72) Inventors: Kelly Valentine, New Britain, CT (US); Matthew Chowaniec, Madison, CT (US); Michael Ingmanson, Stratford, CT (US)

(21) Appl. No.: 15/796,925

(22) Filed: Oct. 30, 2017

Related U.S. Application Data
(60) Provisional application No. 62/417,628, filed on Nov. 4, 2016.

(51) Int. Cl. A61L 2/28; A61L 2/10; A61L 2/24

(52) U.S. Cl. CPC A61L 2/28; A61L 2/10; A61L 2202/24; A61L 2202/11; A61L 2202/14; A61L 2/24

(57) ABSTRACT

A sterilization system includes a sterilization device and a control device. The sterilization device includes at least one emitter configured to emit ultraviolet energy, and at least one localization system configured to determine positional and spatial relation between the sterilization device and a target area. The control device includes a storable medium and a processor in communication with the emitter and the localization system. The control device is configured to scan the target area, via the localization system, execute a sterilization process based on a sterilization profile, via the at least one emitter, extrapolate data received from the localization system in an iterative process, via the processor, and generate a sterilization map of the target area indicative of areas within the target area determined to be sterilized based on the extrapolated data.

### CROSS-REFERENCE TO RELATED APPLICATIONS

This application claims the benefit of and priority to U.S. Provisional Patent Application No. 62/417,628 filed Nov. 4, 2016, the entire disclosure of which is incorporated by reference herein.

### BACKGROUND

Contamination of the operative theater or surgical objects therein, such as, for example, surgical tools, robotic systems, robotic subassemblies, and associated components therewith, continues to be an issue. For the disinfection or sterilization of surface contaminates the application of heat, steam, chemical compounds or solutions, ultrasonic energy, gamma radiation, etc., may be utilized. However, efficient, reliable, and systematic sterilization of the operative theater or surgical objects included therein continues to prove problematic. Problems encountered during sterilization may include the obstructions of certain areas of the operative theater, the necessity for nondestructive sterilization methods to accommodate precision tools and robotic systems, such as, for example, electromechanical devices incapable of withstanding traditional chemicals, steam, or gas sterilization, and the ongoing requirement for frequent, expeditious, and reliable sterilization of the operative theater or surgical objects in a time effective manner.

As a result thereof, there is a need for a time saving, cost efficient, systematic, and reliable sterilization system, which may be operated manually or automatically, for either the operative theater or a surgical object, which additionally prevents inadvertent exposure to clinicians and damage to precision tools.

### SUMMARY

In accordance with an aspect of the present disclosure, a sterilization system including a sterilization device and a control device is provided. The sterilization device includes at least one emitter configured to emit ultraviolet energy and at least one localization system configured to determine positional and spatial relation between the sterilization device and a target area. The control device includes a storable medium and a processor in communication with the emitter and the localization system. The control device is configured to scan the target area via the localization system; execute a sterilization process based on a sterilization profile via the at least one emitter; extrapolate data received from the localization system in an iterative process via the processor; and generate a sterilization map of the target area indicative of areas within the target area determined to be sterilized based on the extrapolated data.

In an embodiment, the control device is further configured to monitor the target area during the sterilization process via the at least one localization system; identify an unsafe target area condition; and issue an unsafe target area alarm via an alarm of the control device when the target area is in the unsafe target area condition. In an embodiment, the control device is further configured to automatically stop the sterilization process based on the issued unsafe target area condition alarm. In a further embodiment, the control device is configured to save the sterilization profile, via the storable medium.

In yet a further embodiment, the control device further includes a user interface in communication with the storable medium and the processor. In a further embodiment, the control device is configured to display, via the user interface, the sterilization map. Further still, in an embodiment, the localization system includes a localization emitter, a localization receiver, and a camera. In yet a further embodiment, the control device is configured to overlay the sterilization map onto an image from the camera via the user interface.

In a further embodiment, the control device is configured to generate the sterilization profile based on operational parameters of the sterilization system. Further still, in an embodiment, the operational parameters are input, via the user interface, or retrieved, via the storable medium. In yet a further embodiment, the operational parameters includes at least one of an ultraviolet dose, an ultraviolet intensity, an ultraviolet exposure time, dwell time, sterilization start and stop times, sterilization frequency over a period of time, or a sterilization schedule. Further still, in an embodiment, the control device is configured to initiate the sterilization process automatically based on the operational parameters.

In another embodiment, the sterilization system further includes a movable unit, where the sterilization device is supported thereon, and the at least one emitter and the at least one localization system are disposed externally thereof. In a further embodiment, the movable unit further supports the control device. In yet a further embodiment, the control device is configured to determine an optimal position of the sterilization device, with respect to the target area, based on an iterative process of the generated sterilization map, and recommend the determined optimal position of the sterilization device.

In yet another embodiment, the sterilization device further includes an arm supporting the at least one emitter. The arm is configured to articulate with respect to the target area, and the control device is configured to remotely direct articulation thereof. In a further embodiment, articulation of the arm is based on the generated sterilization map, such that the at least one emitter is articulated with respect to the target area to maximize sterilization thereof. In another embodiment, each of the sterilization device and the control device further include a wireless communication device configured for wireless communication therebetween.

In accordance with another embodiment of the present disclosure a method of sterilizing a target area includes providing a sterilization device and a control device. The sterilization device includes at least one emitter configured to emit ultraviolet energy, and at least one localization system configured to determine positional and spatial relation between the sterilization device and the target area. The control device includes a storable medium, and a processor, where the storable medium and the processor are in communication with the at least one emitter and the at least one localization system. The method includes scanning a target area via the at least one localization system. The method further includes executing a sterilization process based on a sterilization profile via the at least one emitter. Further, the method includes extrapolating data received from the localization system in an iterative process via the processor. The method also includes generating a sterilization map of the target area indicative of areas within the target area determined to be sterilized based on the extrapolated data.

In an embodiment, the method further includes determining identification and characteristics information of the target area, and updating the sterilization profile based on the determined identification and characteristic information of the target area.

In yet another embodiment, scanning the target area further includes identifying a safe target area condition, and executing the sterilization process further includes initiating the sterilization process if the target area is in the safe target area condition.

In another embodiment, the method further includes providing a user interface in communication with the sterilization device and the control device, and displaying the generated sterilization map via the user interface. In a further embodiment, providing the sterilization device further includes providing the localization system including a localization emitter, a localization receiver, and a camera. In yet a further embodiment, displaying the generated sterilization map further includes overlaying the sterilization map onto an image from the camera.

In yet another embodiment, the method further includes generating the sterilization profile based on operational parameters of the sterilization system. The operational parameters include at least one of an ultraviolet dose, an ultraviolet intensity, an ultraviolet exposure time, dwell time, sterilization start and stop times, sterilization frequency over a period of time, or a sterilization schedule.

In another embodiment, the method includes providing a movable unit supporting the sterilization device. The method also includes determining an optimal position of the sterilization device with respect to the target area based on an iterative process of the generated sterilization map and recommending the determined optimal position of the sterilization device.

According to yet another aspect of the present disclosure, a sterilization system is provided and includes a sterilization device having an ultraviolet energy emitter; and a localization system for determining at least one of positional or spatial relations between the sterilization device and a target area.

The sterilization system further includes a control device having a storable medium and a processor in communication with the ultraviolet energy emitter and with the localization system. The control device is configured to use the localization system to scan the target area; execute a sterilization process based on a sterilization profile via the ultraviolet energy emitter; extrapolate data received from the localization system in an iterative process via the processor; and generate a sterilization map of the target area indicative of areas within the target area determined to be sterilized based on the extrapolated data.

### BRIEF DESCRIPTION OF THE DRAWINGS

Embodiments of the present disclosure are described herein with reference to the accompanying drawings, wherein:

FIG. 1A is a schematic illustration of a sterilization system in accordance with the present disclosure, illustrating an exemplary embodiment of a sterilization device and a control device;

FIG. 1B is a schematic illustration of the sterilization system of FIG. 1A, illustrating a further embodiment of the sterilization device and the control device; and

FIGS. 2A and 2B are flow charts illustrating an exemplary method of operating the sterilization system of FIG. 1A or FIG. 1B in accordance with the present disclosure.

### DETAILED DESCRIPTION

As will be described in detail below, embodiments of the present disclosure describe a sterilization system which may be used to sterilize, either individually or simultaneously, an operative theater or a surgical object. As used herein, a surgical object “SO” may include, for example, a surgical tool or device, a patient monitoring system, a patient restraining device, a robotic system, a robotic subassembly, or any other associated component utilized within the operating theater. As used herein, a target area “TA” of the sterilization system includes the surface area of a desired location, which may include a surgical object “SO”, that will undergo a sterilization process, and a surrounding area “SA” of the sterilization system includes the area immediately surrounding a sterilization device of a sterilization system, as descried herein below, and encompasses the target area “TA.” The sterilization system and methods thereof are described in detail with reference to the drawings, in which like reference numerals designate identical or corresponding elements in each of the several views.

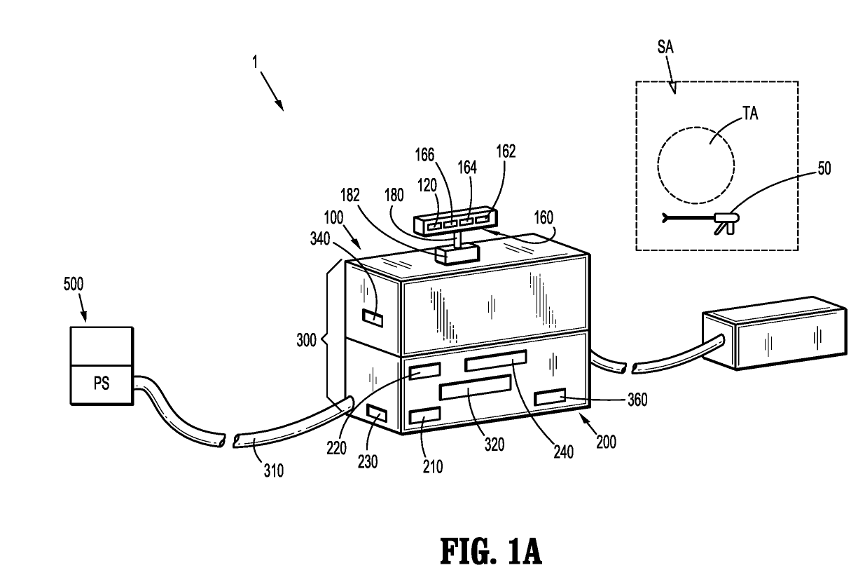

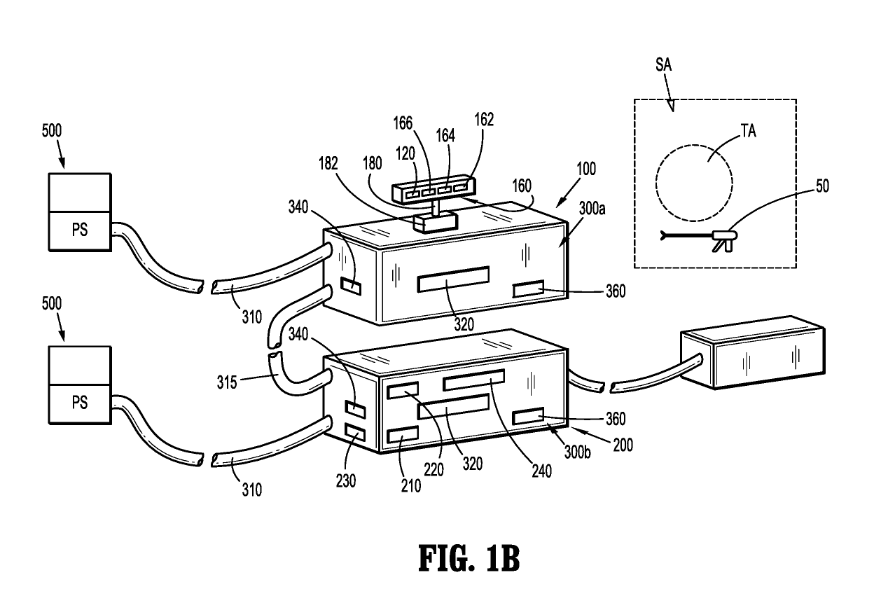

With reference to FIG. 1A, an exemplary embodiment of a sterilization system in accordance with the present disclosure, sterilization system 1, includes a sterilization device 100 and a control device 200. Sterilization device 100 and control device 200 are in communication such that control device 200 may execute and control sterilization device 100 to perform sterilization of the target area “TA” and/or a surgical object “SO” within the target area “TA.” Sterilization device 100 and control device 200 may be contained in a single housing to form a single unit 300. Unit 300 may be stationary or movable, either manually or automatically, as discussed below.

With sterilization device 100 and control device 200 supported in the housing of unit 300, a single cable 310 may be utilized to provide power thereto such that housing 300 is interconnected with a power source “PS”, such as, for example, an AC power source, a DC power source, a battery, a generator, or any other power source known in the art. It is envisioned that cable 310 may be selectively removable from unit 300, wherein unit 300 may further include a rechargeable power source 320 such that surgical system 1 may operate independent of cable 310 and without direct connection with power source “PS.” Cable 310 may additionally be configured to transfer data between sterilization system 1 and one or more external computing devices 500, such as, for example, a computer, a server, a remote user interface, etc. Unit 300 may further include a wireless communication device 340, such as, for example, Wifi®, BlueTooth®, or any other wireless communication devices as is known in the art, such that data may be transferred wirelessly between sterilization device 100 and control device 200, supported in the housing of unit 300, and the external computing device 500.

With reference to FIG. 1B, sterilization device 100 and control device 200 may alternatively be supported individually in a housing of a unit 300a, 300b, respectively. Sterilization device 100 and control device 200 may be configured to be coupled together or positioned independently at remote locations with respect to one another. One or both of sterilization device 100 or control device 200 may be configured to be movable, either manually or automatically, as discussed herein. It should be appreciated that inadvertent exposure to UV radiation may be reduced or avoided by positioning control device 200 at a remote location with respect to sterilization device 100. In such an embodiment, sterilization device 100 and/or control device 200 may be individually interconnected to power source “PS” or external computing device 500, via a respective cable 310. Sterilization device 100 and control device 200 may be interconnected for the transfer of data therebetween via a cable 315, where cable 315 may additionally provide for the transfer of power therebetween such that only one of sterilization device 100 or control device 200 requires cable 310 for connection with power source “PS.”

It is further envisioned that sterilization device 100 and/or control device 200 may include rechargeable power source 320, as discussed herein. Further, sterilization device 100 and/or control device 200 may include a respective wireless communication device 340, such that sterilization device 100 and control device 200 may wirelessly transfer data therebetween, independent of cable 315, and may further, individually or simultaneously, transfer data wirelessly between external computing device 500.

Sterilization device 100 includes an ultraviolet radiation emitter (hereinafter “UV emitter”) 120 and a localization system 160 supported on the housing of unit 300, of FIG. 1A, or the housing of unit 300a, of FIG. 1B. UV emitter 120 is configured to emit short-wavelength ultraviolet light for a time and intensity sufficient that microorganisms, exposed thereto, are killed or rendered inactive, thus leaving them unable to perform vital cellular functions. Through activation of UV emitter 120, target area “TA” within the surrounding area “SA” of sterilization device 100 may be effectively sterilized. UV emitter 120 may be a UV emitting LED or light bulb, such as, for example, a mercury-vapor lamp, an amalgam UV lamp, a low-pressure UV lamp, a medium-pressure UV lamp, or any other UV emitting source known in the art configured to continuously or intermittently emit UV light in a wavelength range of approximately 100 nm to 280 nm in order to achieve a desired level of sterilization.

For surface sterilization, UV effectiveness may be estimated by calculating the UV dose which is delivered to the microbial population of the target area “TA”. UV dose represents the amount of UV irradiation absorbed by an exposed population of microbes, and may be represented in terms of “microwatt seconds per square centimeter” (μWs/cm²) and may be calculated as follows:

UV Dose μWs/cm²=(UV Intensity μW/cm²)×(Exposure Time (s)).

To achieve 90% inactivity of most bacteria and viruses a UV dose between approximately 2,000 μWs/cm² and 8,000 μWs/cm² is desired, where larger parasites may require larger UV doses.

Localization system 160 of sterilization device 100 is configured to cooperatively act with control device 200 to determine the positional and spatial relation of sterilization device 100 with respect to the target area “TA” or the surrounding area “SA.” More particularly, localization system 160 and control device 200 act to extrapolate a position of the sterilization device 100 with respect to the target area “TA”, identify a safe or unsafe sterilization condition within the surrounding area “SA”, and determine an opportune position of sterilization device 100 with respect to the target area “TA” to achieve the desired level of sterilization. Localization system 160 optically determines the positional and spatial relation of sterilization device 100, and includes a localization source 162 and a localization receiver 164. Localization source 162 cooperatively acts with localization receiver 164 to emit and receive an optical signal, respectively. Localization source 162 may include an infra-red emitter, an LED diode, or a laser diode, where localization receiver 164 may include any corresponding receiver, or any further combination as is known in the art for positional and spatial determinations. Localization system 160 may further include a camera 166 configured to provide photographic or video visualization of the target area “TA” and the surrounding area “SA” to the clinician. The visualization of the target area “TA” may be displayed to the clinician or incorporated with data retrieved from localization source 162 and localization receiver 164, via control device 200, when determining the positional and spatial relation of sterilization device 100. It is further envisioned that localization system 160 may additionally or alternatively include acoustic, sonar, radar, or other known means in the art to determine the positional and spatial relation of sterilization device 100.

Sterilization device 100 may include a plurality of UV emitters 120 or localization systems 160, e.g., a plurality of localization sources 162, a plurality of localization receivers 164, or a plurality of cameras 166, to facilitate 360 degrees of sterilization, positional and spatial relation, and visualization. Unit 300 or sterilization device 100 may further include an articulating arm 180 extending therefrom. In such an embodiment, UV emitter 120 and/or localization system 160 may be supported on an arm or stand 180 (e.g., articulating, pivoting, etc.) to further enhance 360 degrees of sterilization, positional and spatial relation, and visualization. A clinician may manually articulate articulating arm 180 or control device 200 and/or external computing device 500 may direct remote articulation of articulating arm 180 using a drive mechanism 182 supported on housing 300 or housing 300a. Drive mechanism 182 may include one or more motors, servos, linkages, drive belts, gears, cables, or any other mechanism known in the art to achieve articulation of articulation arm 180. Drive mechanism 182 is coupled to articulating arm 180 and is in communication with control device 200 and/or external computing device 500.

Further still, sterilization device 100 and/or control device 200 may be configured for adjustment and repositioning with respect to target area “TA.” Unit 300, or sterilization device 100 and/or control device 200, may include wheels or casters configured to facilitate movement about the operative theater, and may be further configured to traverse along a track or guide system. It is envisioned that adjustment and repositioning of sterilization device 100 and/or control device 200 may be manually directed by the clinician, or may be configured for automated control or operation. In such an embodiment, unit 300, sterilization device 100, or control device 200 further include a drive system 360 in communication therewith or with external computing device 500, such that the position of sterilization device 100 and/or control device 200 may be remotely adjusted or repositioned.

Control device 200 includes a processor 210, a storable medium 220, and a data exchange interface 230, such as, for example, a USB port, a disk drive, etc. Control device 200 may further include a user interface 240 which displays information to, and receives inputted information from the clinician, such as the visualization of the target area “TA” provided by camera 166. It is envisioned that control device 200 may receive identification or characteristic data specific to the target area “TA” such as an operating room or surgical area having specific dimensions and obstructions therein, the surgical object “SO”, such as a surgical tool having stringent sterilization requirements and/or limitations, or the surrounding area “SA.” The target area “TA”, surgical object “SO”, or surrounding area “SA” data may be provided by a clinician via user interface 240 or external computing device 500 or may be automatically determined by control device 200. In such an embodiment, target area “TA”, surgical object “SO”, or the surrounding area “SA” may include a Radio-Frequency Identification tag, a Near Field Communication chip, or any similar identifying communication device as is known in the art such that control device 200 may identify target area “TA”, surgical object “SO”, or surrounding area “SA” and may retrieve characteristic data provided therefrom or previously stored on storable medium 220 pertaining thereto.

Control device 200 executes a sterilization profile which directs and controls the sterilization process of sterilization device 100. The sterilization profile is a compilation of operational parameters of sterilization system 1 specific to the desired sterilization process, and may include any of the following: UV Dose, UV intensity, UV exposure time, dwell time, desired sterilization start and stop times, desired sterilization frequency over a period of time, date and time stamps for executed sterilization processes, identification or characteristics of the target area “TA”, identification or characteristics of the surgical object “SO”, or characteristics of the surrounding area “SA.” Individual operational parameters or a sterilization profile may be retrieved from storable medium 220, input by a clinician via user interface 240, or transferred to control device 200 via external computing device 500.

Further, control device 200 may independently generate a sterilization profile, or update an existing sterilization profile, based on: the positional and spatial relation of sterilization device 100 with respect to the target area “TA”, via localization system 160; inputted operational parameters; or the identification and characteristic data received from the target area “TA”, surgical object “SO”, or surrounding area “SA.” It should be appreciated that the operational parameters, sterilization profile being executed, and any saved sterilization profiles may be displayed on user interface 166 or transmitted to external computing device 500. Control device 200 may additionally be configured to automatically initiate the sterilization process based on a sterilization schedule which may be incorporated into the sterilization profile. The sterilization schedule may include a planned date, time, or frequency for sterilization, which may be saved on storable medium 220 or provided to control device 200, via user interface 240 or external computing device 500.

To achieve the desired degree of sterilization a clear line of sight between sterilization device 100 and the target area “TA” is desired. UV radiation emitted by UV emitter 120 is inhibited by physical obstructions, which may create blind or obstructed spots, thus preventing the desired degree of sterilization from being achieved. It is envisioned that sterilization system 1 may include one or more mirrors to overcome obstructions in the target area “TA” to redirect UV radiation emitted by UV emitter 120. Additionally, control device 200 may be configured to analyze the target area “TA”, recognize obstructed spots within the target area “TA”, and identify areas where the desired level of sterilization may not be achieved.

More particularly, control device 200 generates a sterilization map of the target area “TA.” Control device 200 generates the sterilization map by extrapolating, in an iterative process via processor 210, the sterilization profile being executed and the data received from the localization system 160. The sterilization map is indicative of locations within the target area “TA” determined to be sterilized to the desired level of sterilization, and locations within the target area “TA” determined to be obstructed, and thus not sterilized to the desired level of sterilization. Based on the identified obstructed areas, the sterilization map indicates to the clinician areas which require manual sterilization. The sterilization map may be directly displayed on the user interface 240, overlaid over the visualization provided by camera 166 and displayed on the user interface 240, transmitted to the external computing device 500, and/or saved via the storable medium 220.

Based on the sterilization map, the clinician may determine an optimal initial or subsequent position for sterilization device 100 with respect to the target area “TA”, and further, may utilize the sterilization map as visual indicia to verify which portions of the target area “TA” have been, or will be, sterilized to the desired level of sterilization. It should be appreciated that the sterilization map may be presented to the clinician prior to, during, or at the conclusion of the sterilization process, and that the clinician may adjust or reposition sterilization device 100 either by repositioning housing 300 or housing 300a, or alternatively by adjusting articulating arm 180.

It is envisioned that control device 200, via an iterative process, may determine and recommend where sterilization device 100 should be positioned or repositioned during one or more subsequent sterilization processes to achieve an optimal and desired level of sterilization. Such a recommendation may be displayed on user interface 240 or transmitted to external computing device 500. It is further envisioned that control device 200 may automatically reposition sterilization device 100, via drive assembly 360 of housing 300 or housing 300a, or automatically adjust articulating arm 180, via drive mechanism 182, to achieve the desired level of sterilization of the target area “TA.”

Control device 200 may be further configured to monitor the surrounding area “SA” and target area “TA” during the sterilization process, via the localization system 160, such that a safe and unsafe target area “TA” condition may be identified and maintained based on sensed motion. To ensure safety to clinicians during active sterilization, control device 200 continuously or intermittently monitors the surrounding area “SA” to ensure the target area “TA” is clear of clinicians. Upon detection of a clinician in proximity to the target area “TA”, control device 200 determines an unsafe target area condition and initiates an alarm and/or immediately stops the sterilization process. The unsafe target area condition alarm may be an audible or visual indicator which is emitted from or displayed on user interface 240 or external computing device 500.

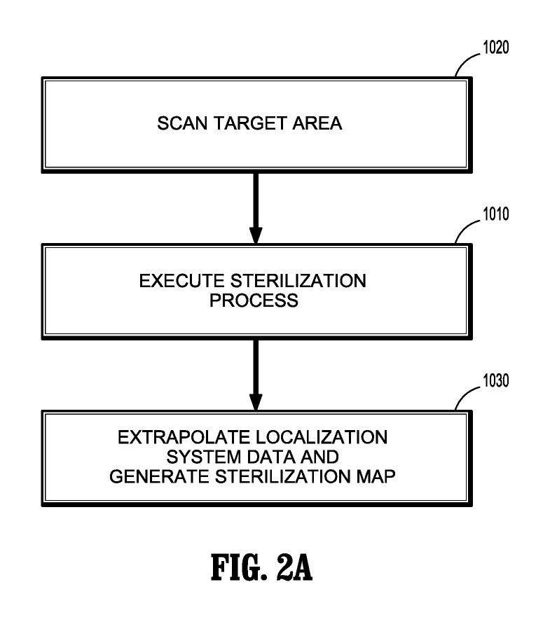

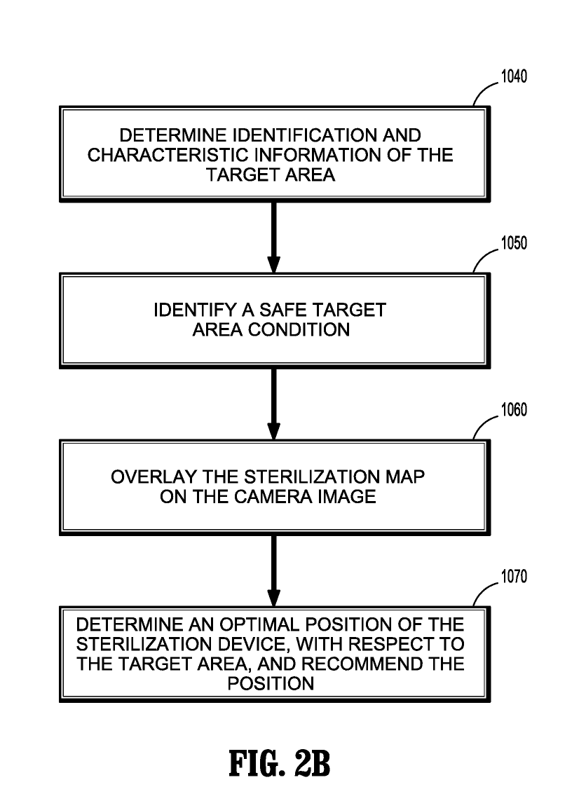

With reference to FIG. 2A, an exemplary method of performing sterilization in accordance with the present disclosure is illustrated. At step 1010, control device 200 scans the target area “TA.” At step 1020, control device 200 executes the sterilization process based on the sterilization profile. The sterilization profile may be provided by the clinician, retrieved from storable medium 220, received from external computing device 500, or generated by control device 200 based on the operational parameters of the desired sterilization process. At step 1030, control device 200 extrapolates data received from localization system 160 and generates a sterilization map of the target area “TA.”

With reference to FIG. 2B, the method may further include, at step 1040, determining identification and characteristic information of the target area “TA”, and updating the sterilization profile based thereon. To ensure safety of the surrounding area “SA”, at step 1050, control device 200 may identify a safe target area “TA” condition, execute the sterilization process only if the safe target area condition is verified or maintained, and issue an alarm or stop the sterilization process if an unsafe target area condition is determined. Utilizing user interface 240, at step 1060, control device 200 may overlay the sterilization map and the visualization from the camera 166. At step 1070, control device 200 may determine and recommend the optimal position of sterilization device 100, with respect to the target area.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

1. A sterilization system comprising:
a sterilization device including:
at least one emitter configured to emit ultraviolet energy; and
at least one localization system configured to determine positional and spatial relation between the sterilization device and a target area;
a control device including a storable medium and a processor in communication with the emitter and the localization system, the control device configured to:
scan the target area via the localization system;
execute a sterilization process based on a sterilization profile via the at least one emitter;
extrapolate data received from the localization system in an iterative process via the processor; and
generate a sterilization map of the target area indicative of areas within the target area determined to be sterilized based on the extrapolated data.

2. The sterilization system of claim 1, wherein the control device is configured to:
monitor the target area during the sterilization process, via the at least one localization system;
identify an unsafe target area condition; and
issue an unsafe target area alarm, via an alarm of the control device, when the target area is in the unsafe target area condition.

3. The sterilization system of claim 2, wherein the control device is configured to automatically stop the sterilization process based on the issued unsafe target area condition alarm.

4. The sterilization system of claim 1, wherein the control device is configured to save the sterilization profile via the storable medium.

5. The sterilization system of claim 1, wherein the control device further includes a user interface in communication with the storable medium and the processor.

6. The sterilization system of claim 5, wherein the control device is configured to display, via the user interface, the sterilization map.

7. The sterilization system of claim 6, wherein the localization system includes, a localization emitter, a localization receiver, and a camera.

8. The sterilization system of claim 7, wherein the control device is configured to overlay the sterilization map onto an image from the camera, via the user interface.

9. The sterilization system of claim 5, wherein the control device is configured to generate the sterilization profile based on operational parameters of the sterilization system.

10. The sterilization system of claim 9, wherein the operational parameters are input, via the user interface, or retrieved, via the storable medium.

11. The sterilization system of claim 10, wherein the operational parameters including at least one of an ultraviolet dose, an ultraviolet intensity, an ultraviolet exposure time, dwell time, sterilization start and stop times, sterilization frequency over a period of time, or a sterilization schedule.

12. The sterilization system of claim 11, wherein the control device is configured to initiate the sterilization process automatically based on the operational parameters.

13. The sterilization system of claim 1, further comprising a movable unit, the sterilization device supported thereon, the at least one emitter and the at least one localization system disposed externally thereof.

14. The sterilization system of claim 13, wherein the movable unit further supports the control device.

15. The sterilization system of claim 13, wherein the control device is configured to:
determine an optimal position of the sterilization device with respect to the target area, based on an iterative process of the generated sterilization map; and
recommend the determined optimal position of the sterilization device.

16. The sterilization system of claim 1, wherein the sterilization device further includes an arm, the at least one emitter supported thereon, the arm configured to articulate with respect to the target area, and the control device configured to remotely direct articulation thereof.

17. The sterilization system of claim 16, wherein articulation of the arm is based on the generated sterilization map, such that the at least one emitter is articulated with respect to the target area to maximize sterilization thereof.

18. The sterilization system of claim 1, wherein each of the sterilization device and the control device further include a wireless communication device configured for wireless communication therebetween.

19. A sterilization system comprising:
a sterilization device including:
an ultraviolet energy emitter; and
a localization system for determining at least one of positional or spatial relations between the sterilization device and a target area;
a control device including a storable medium and a processor in communication with the ultraviolet energy emitter and with the localization system, the control device configured to:
use the localization system to scan the target area;
execute a sterilization process based on a sterilization profile via the ultraviolet energy emitter;
extrapolate data received from the localization system in an iterative process via the processor; and
generate a sterilization map of the target area indicative of areas within the target area determined to be sterilized based on the extrapolated data.
